(19) United States
(12) Patent Application Publication
(10) Pub. No.: US 2003/0045725 A1
(43) Pub. Date: Mar. 6, 2003

(54) Method for the production of aromatic or heteroaromatic aldehydes or ketones by oxidative decarboxylation

(76) Inventors: Wolfram Hendel, Leonding (AT); Ferdinand Heu, Linz (AT); Peter Poechlauer, Linz (AT); Berthold Winkler, Steyr (AT)

Correspondence Address: WENDEROTH, LIND & PONACK, L.L.P., 2033 K STREET N. W., SUITE 800, WASHINGTON, DC, 20006-1021, US

(21) Appl. No.: 10/239,075

(22) PCT Filed: Mar. 21, 2001
(86) PCT No.: PCT/EP01/03222

(51) Int. Cl. C07D 213/46; C07C 321/26; C07C 247/18; C07C 223/06; C07C 45/27

(52) U.S. Cl. 546/315; 552/6; 562/459; 564/328; 568/41; 568/306; 568/319

(57) ABSTRACT

A method for the production of substituted aromatic aldehydes or ketones and optionally substituted heteroaromatic aldehydes or ketones of formula (I) by reacting a compound of formula (II) in a suitable solvent in the presence of a carbonyl compound of formula (III), optionally in the presence of oxygen at a normal pressure or high temperature and at temperatures of 5-200° C.

Aromatic aldehydes and ketones are important intermediates in the chemical, pharmaceutical and cosmetic industries, and the literature already discloses a series of processes for preparing them which are, however, unsatisfactory for industrial utilization.

As well as the variants of the Reimer-Tiemann reaction (Chem. Rev. 60 (1960), 169) these are effected, for example under pressure and using transition metal catalysts or cyclodextrins, by hydroxymethylating phenol with subsequent oxidation, and also by the reaction of phenol with glyoxylic acid and subsequent oxidative decarboxylation of the mandelic acid derivatives obtained as intermediates in the presence of metal salts (for example U.S. Pat. No. 2,640,083).

Chem. Abstr. (1982): 597981 discloses the oxidation of hydroxyphenylglycine to hydroxybenzaldehyde using metal catalysts. However, as comparative experiments showed, the reaction is very difficult to conduct owing to the high proportion of by-products.

Tetrahedron Letters No. 24, pp. 2139-2140 (1971) discloses a process for oxidatively decarboxylating aliphatic 60 -amino acids, in particular leucine and valine, using isatogens, such as isatin or ninhydrin, to the corresponding aldehydes and isatogen reduction products. However, when other amino acids are used, such as phenylalanine, the paper discloses that tarry or oily by-products are obtained.

It has now been found, unexpectedly that, aromatic amino acids which are substituted on the aromatic and optionally substituted heteroaromatic amino acids may be converted using specific carbonyl compounds as catalysts in combination with oxygen or in molar quantities to the corresponding aldehydes and ketones in high yield and purity.

The invention accordingly provides a process for preparing substituted, aromatic aldehydes or ketones and optionally substituted, heteroaromatic aldehydes and ketones of the formula

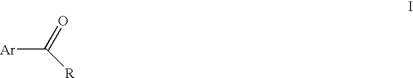

where Ar is a mono- or polysubstituted aromatic or an optionally mono- or polysubstituted heteroaromatic radical and the substituents are OH, C₁-C₆-alkyl, C₁-C₆-alkoxy, C₁-C₆-carboxylic acids or esters having 1-6 carbon atoms in the ester moiety, methylenedioxy, phenyl, halogen, SO₃H, NO₂, N₃, NR₁R₂ or SR₁ where R₁ and R₂ are each independently H, phenyl or C₁-C₆-alkyl, and R is H or an unsubstituted or polysubstituted alkyl group having 1-12 carbon atoms, unsubstituted or substituted benzyl or diphenylmethyl, and the substituents are heteroaromatics, halogens, NO₂, N₃, SO₃H, NR₁R₂ or SR₁ where R₁ and R₂ are each independently H, phenyl or C₁-C₆-alkyl, characterized in that a compound of the formula

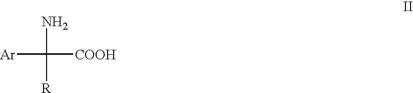

where Ar and R are each as defined above is converted in a suitable solvent in the presence of a carbonyl compound of the formula

R₃C(O)(CH═CH)ₙC(O)(X)_{y}R₃ III

where n is 0 or an integer from 1 to 3, y is 0 or 1, X is NR₁, O or SO₂ and R₁ is H, C₁-C₆-alkyl or CH₂COOH, R₃ and R₃ are each independently H, OH, C₁-C₆-alkyl, C₁-C₆-alkoxy or phenyl or together form a (—C═C—)ₘ bond where m is 1 or 2, or a fused ring system which is optionally substituted by OH, C₁-C₆-alkyl, C₁-C₆-alkoxy, C₁-C₆-carboxylic acids or esters having 1-6 carbon atoms in the ester moiety, phenyl, halogen, SO₃H, NO₂, N₃, NR₁R₂ or SR₁ where R₁ and R₂ are each independently H, phenyl or C₁-C₆-alkyl, and may optionally contain a heteroatom substituted by C₁-C₆-alkyl or CH₂COOH, to the corresponding aldehyde or ketone of the formula I, optionally in the presence of oxygen, and at atmospheric or elevated pressure and at temperatures of from 5 to 200° C.

The process according to the invention converts compounds of the formula II to the corresponding aldehydes or ketones of the formula I.

Useful compounds of the formula II are α-amino acids which have a substituted aromatic or an optionally substituted heteroaromatic Ar radical.

Aromatic and heteroaromatic Ar radicals are radicals which are derived from aromatics or heteroaromatics having one or more heteroatoms or from fused ring systems which optionally have one or more heteroatoms, such as benzene, pyrrole, furan, thiophene, pyridine, pyran, thiopyran, pyrimidine, indene, imidazole, pyrazole, thiazole, oxazole, naphthalene, anthracene, quinoline, isoquinoline, benzo(g)isoquinoline, indole, coumaron, thionaphthene, acridine, etc. The aromatic radicals are substituted by one or more substituents from the group of OH, methylenedioxy, linear, branched or cyclic C₁-C₆-alkyl- or C₁-C₆-alkoxy radicals, C₁-C₆-carboxylic acids or esters having 1-6 carbon atoms in the ester moiety, phenyl, halogen, SO₃H, NO₂, N₃, NR₁R₂ and SR₁ where R₁ and R₂ are each independently H, phenyl or a linear, branched or cyclic C₁-C₆-alkyl radical. Heteroaromatic radicals may be unsubstituted or substituted by one or more of the above substituents.

Preference is given to Ar being an aromatic radical or a fused ring system having a maximum of one heteroatom, such as phenyl, pyrrolyl, pyridinyl, thiophenyl, naphthyl, etc. Particular preference is given to aromatic radicals having only one ring and a maximum of one heteroatom phenyl, pyrrolyl, pyridinyl, thiophenyl, etc.

Preferred substituents are OH, C₁-C₄-alkoxy, such as methoxy, ethoxy, propoxy and butoxy, and NR₁R₂ where R₁ and R₂ are each H or C₁-C₄-alkyl. Particular preference is given to OH and C₁-C₂-alkoxy.

Very particular preference is given to using p-hydroxyphenylglycine, p-alkoxyphenylglycines such as p-methoxyphenylglycine, α-substituted amino acids such as methylphenylglycine or methylenedioxyphenylglycine, and dihydroxy- or polyhydroxyphenylglycine in the process according to the invention.

The starting compounds may be used as racemates or in an enantiomerically pure form as the R- or S-enantiomer.

The starting compounds of the formula II are converted to the corresponding aldehydes or ketones of the formula I in a suitable solvent.

Suitable solvents are water or aqueous mixtures with organic and/or inorganic acids, such as acetic acid, sulfuric acid, hydrochloric acid, etc., or with glycerol, dioxane or pyridine, which have a pH of 1-12. Preference is given to aqueous mixtures with organic and/or inorganic acids. Particular preference is given to water or a water/acetic acid, water/hydrochloric acid, water/sulfuric acid or water/sulfuric acid/acetic acid mixture.

According to the invention, the starting compounds are oxidized to the corresponding aldehydes or ketones in the presence of a carbonyl compound of the formula III either in catalytic quantities using oxygen or in molar quantities.

In the formula III, n is 0 or an integer from 1 to 3. Preference is given to n being 0 or 1; y being 0 or 1, X being NR₁, O or SO₂ and R₁ being H, a linear, branched or cyclic C₁-C₆-alkyl radical or CH₂COOH. R₃ and R₃′ are each independently H, OH, a linear, branched or cyclic C₁-C₆-alkyl or C₁-C₆-alkoxy radical or phenyl or together form a (—C═C—)ₘ bond where m is 1 or 2, or a fused ring system which is optionally substituted by OH, C₁-C₆-alkyl, C₁-C₆-alkoxy, C₁-C₆-carboxylic acids or esters having 1-6 carbon atoms in the ester moiety, phenyl, halogen, SO₃H, NO₂, N₃, NR₁R₂ or SR₁ where R₁ and R₂ can each be independently H, phenyl or C₁-C₆-alkyl, and may optionally contain a heteroatom substituted by C₁-C₆-alkyl or CH₂COOH. Useful catalysts are accordingly α-ketoacids, α-keto-lactones, α-ketolactams, α-ketoaldehydes, 1,2-diketones, aldehydecarboxylic acids, dialdehydes, dicarboxylic acids or esters thereof, quinones, etc. Examples thereof are glyoxal, phenylglyoxal, methyl-glyoxal, diacetal, oxalic acid, maleic acid, glyoxylic acid, glyoxylic ester, phenylglyoxylic acid, pyruvic acid, pyruvic ester, phenylpyruvic acid, benzil, p-benzoquinone, o-benzoquinone, indigo, isatin, N-methylisatin, isatinsulfonic acid or isatinacetic acid, etc.

Preference is given to using isatin and substituted isatins, such as N-methylisatin, isatinsulfonic acid, diacetyl, glyoxylic acid or esters thereof, phenylglyoxylic acid, pyruvic acid or esters thereof, phenylglyoxal or p-benzoquinone.

Particular preference is given to isatin, N-methylisatin, isatinsulfonic acid, diacetyl, glyoxylic acid, pyruvic acid or p-benzoquinone. The carbonyl compounds may optionally also be used in the form of a salt.

Depending on the type of carbonyl compound, it is used either in combination with oxygen in catalytic quantities of from 1 to 30 mol %, preferably from 1-20%, as is the case, for example, for isatin or isatinsulfonic acid, or in an equimolar quantity, i.e. in a quantity of from 1.1 to 5, preferably from 1.4 to 3.5, mol of carbonyl compound per mole of starting compound.

The carbonyl compound may be used either in homogeneous form or in heterogeneous form, on a suitable support.

The reaction according to the invention is effected, preferably using the carbonyl compound in catalytic quantities, in the presence of oxygen, for example in the form of pure oxygen, in the form of air or in the form of an N₂/O₂ mixture. In this case, air or oxygen or the N₂/O₂ mixture is blown into the reaction mixture during the reaction.

This is advantageous in particular when using isatin or substituted isatins. For example, the isatin acting as an oxidizing agent is regenerated by the oxygen present.

The reaction may be effected at atmospheric pressure or at elevated pressure. Preference is accordingly given to setting a pressure from 1 to 7 bar, more preferably from 1 to 5 bar.

The reaction temperatures in the process according to the invention, depending on the pressure selected, are from 5 to 200° C., preferably from 15 to 150° C. and more preferably from 50 to 100° C.

The desired aldehyde or ketone of the formula I is isolated, depending on its aggregate state, by customary isolation methods, such as extraction, distillation, crystallization, etc.

Preference is given to using the process to prepare aldehydes or ketones of the formula I where Ar is an aromatic five- or six-membered ring having a maximum of one heteroatom or a fused ring system having a maximum of one heteroatom, each of which is substituted by OH, methylenedioxy, C₁-C₄-alkoxy, such as methoxy, ethoxy, propoxy or butoxy, or NR₁R₂ where R₁ and R₂ are each H or C₁-C₄-alkyl, more preferably by OH or C₁-C₂-alkoxy, and R is H or an unsubstituted or polysubstituted alkyl group having 1-12 carbon atoms, unsubstituted or substituted benzyl or diphenylmethyl, and the substituents are heteroatoms, halogens, NO₂, N₃, SO₃H, NR₁R₂ or SR₁ where R₁ and R₂ are each independently H, phenyl or C₁-C₆-alkyl.

Very particular preference is given to preparing hydroxy- or polyhydroxybenzaldehydes, such as 4-hydroxybenzaldehyde and 3,4-dihydroxybenzaldehyde, or alkoxybenzaldehydes, such as p-methoxybenzaldehyde, or ketones, such as acetophenone.

The aldehydes and ketones of the formula I are obtained in high yields and in high purity.

### EXAMPLE 1

### Reaction of 4-hydroxyphenylglycine with Isatin and Oxygen

4.9 g of 4-hydroxyphenylglycine (29.3 mmol) and 0.77 g of isatin (5.2 mmol) were dissolved in 50 ml of 50% acetic acid at 90° C. Air was then passed in for 15.5 hours with vigorous stirring. After the end of the reaction, the mixture was diluted with three times its volume of water and extracted using MTBE (3×100 ml). The combined organic phases were washed with NaHCO₃ in water. 2.4 g of 4-hydroxybenzaldehyde were obtained as a red-brownish product (yield: 68%; purity: 77% by weight).

### EXAMPLE 2

### Reaction of 4-hydroxyphenylglycine with Isatin-5-sulfonic Acid

In a 250 ml Schmizo jacketed vessel equipped with a stirrer and jacketed coil condenser, 16.67 g of 4-hydroxyphenylglycine (100 mmol) and 2.85 g of sodium isatin-5-sulfonate dihydrate (10 mmol) were stirred in a mixture of 84 ml of acetic acid and 150 ml of water at 90° C., and air (18 hours, 150-200 ml/min) was bubbled via an inlet tube into the red-colored clear reaction solution. The end product was isolated by means of extraction using MTBE (3×100 ml), washing the combined organic phases with NaHCO₃ solution and concentrating on a Rotavapor. 9.7 g of 4-hydroxy-benzaldehyde were obtained as a beige, pulverulent solid (79.4% yield) in a purity of 97.3% by weight.

### EXAMPLE 3

### Sulfonation of Isatin and Use of the Product as Catalyst for Oxidizing 4-hydroxyphenylglcyine

In a 50 ml 2-neck round-bottomed flask equipped with a magnetic stirrer, 3.0 g of isatin (20.4 mmol) were added in portions to 6.0 ml of conc. sulfuric acid (107 mmol) at room temperature and the resulting dark red viscous solution was stirred at 80° C. After complete conversion (3.5 hours), 4.17 g of this solution (corresponds to about 6 mmol of isatinsulfonic acid) were added slowly to 50 ml of water in a 100 ml Schmizo double-jacket vessel equipped with a jacketed coil condenser 50 ml of conc. acetic acid and 10.3 g of 4-hydroxyphenylglycine (60 ml) were added continuously to this solution which was heated to 90° C. and oxygen (30-40 ml/min) was passed in. After about 20 hours, extraction was effected using MTBE (3×100 ml). The combined organic phases were washed with NaHCO₃ solution and concentrated on a Rotavapor. 4-Hydroxybenzaldehye was obtained as a beige product in a purity of 97.0% by weight (51% isolated).

### EXAMPLE 4

### Reaction with Pyruvic Acid

2.6 g of pyruvic acid (29.5 mmol) were added to a solution of 3.3 g of 4-hydroxyphenylglycine (19.8 mmol) in 50 ml of water and stirred at 80° C. for six hours. The product was extracted using MTBE (3×50 ml), and the organic phase washed with NaHCO₃ solution and water, and concentrated on a rotary evaporator.

1.5 g of 4-hydroxybenzaldehyde were obtained as a beige-colored product (62% yield) in a purity of 92.6% by weight.

### EXAMPLE 5

### Reaction with Glyoxylic Acid

4.5 g of a 50% strength solution of glyoxylic acid solution (30.6 mmol) were added to a solution of 3.4 g of 4-hydroxyphenylglycine (20.5 mmol) in 50 ml of water at 90° C. and stirred for 3 hours. The product was extracted using MTBE (3×50 ml), and the organic phase washed with NaHCO₃ solution and water, and concentrated on a rotary evaporator.

0.93 g of 4-hydroxybenzaldehyde were obtained as a beige-colored product (37.2% yield) in a purity of 98.5% by weight.

### EXAMPLE 6

### Reaction with Diacetyl

In a 250 ml three-necked flask, 19.9 g of 4-hydroxyphenylglycine (119 mmol) were stirred in 100 ml of water, adjusted to pH 1 using conc. hydrochloric acid (6.5 ml) and heated to 80° C. 26.3 g of diacetyl (239 mmol) were added to the clear solution. After 2.5 hours, the hydroxyphenylglycine was completely converted. The solution was diluted using water (50 g), extracted 3×using MTBE (100-150 ml) and the combined organic phases were concentrated on a rotary evaporator. The viscous residue was taken up using water (20 ml) and heated and the black oil which separates removed (at 60-70° C.). From the aqueous solution, colorless crystals precipitated: 3.2 g in a purity of 96.6% by weight. Two further fractions were recovered from the mother liquor (0.78 g; 94.2% by weight and 1.32 g; 84.2% by weight). Total yield: 5.3 g (36%).

### EXAMPLE 7

### Reaction of S-methylphenylglycine with Isatinsulfonic Acid and Oxygen

A mixture of 16.5 g (100 mmol) of S-α-methyl-phenylglycine and 2.5 g (10 mmol) of sodium isatin-sulfonate in 170 ml of dilute acetic acid (50% strength) was stirred at 90° C. Oxygen was passed into the mixture for 31 hours. After cooling to room temperature, the mixture was extracted using 2×100 ml of MTBE, the MTBE phases were combined and washed to neutrality using 200 ml of dilute KOH. After withdrawing the solvent under reduced pressure, a slightly colored residue (5.6 g) having an acetophenone content of 80% was obtained (yield 36%).

### EXAMPLE 8

### Reaction of S-methylphenylglycine with Isatin

A mixture of 49.6 g (300 mmol) of S-α-methyl-phenylglycine and 4.4 g (30 mmol) of isatin in 500 g of dilute sulfuric acid (3%) was stirred at 85° C. Oxygen was passed into the mixture for 17 hours. After cooling to RT, the mixture was extracted using 2×200 ml of MTBE and the resulting acetophenone purified by distillation. (34% yield, purity: 98 area % by GC)

### EXAMPLE 9

### Reaction of 3,4-dihydroxyphenylglycine with Isatinsulfonic Acid

A mixture of 18.3 g (100 mmol) of 3,4-dihydroxyphenylglycine and 2.5 g (10 mmol) of sodium isatinsulfonate in 150 ml of water and 6.2 g of conc. sulfuric acid was stirred at 95° C. Oxygen was passed into the mixture for 14 hours. After cooling to RT, the mixture was extracted using 2×100 ml of MTBE, the MTBE phases were combined and washed to neutrality using 200 ml of dilute KOH. After withdrawing the solvent under reduced pressure, 3,4-dihydroxybenzaldehyde was obtained (yield 66%).

### Comparative Experiment 1: Reaction of 4-hydroxyphenylglycine with CUCl₂ and O₂:

(comparison to Chem. Abstr. (1982): 597981)

In a 250 ml jacketed vessel, 10.0 g of 4-hydroxyphenylglycine (60 mmol) and 0.8 g of copper(II) chloride (6 mmol) were dissolved in 200 ml of water and adjusted to pH=10 using 10% sodium hydroxide solution (46 g). At 94° C., oxygen was passed in (30 ml/min) for 15 hours. HPLC analysis after 15 hours showed 41% by weight of 4-hydroxybenzaldehyde at 100% conversion. Workup by extraction using MTBE (4×100 ml) gave 1.83 g (25%) of 4-hydroxybenzaldehyde in a purity of 87% by weight.

Comparative Example 2: Reaction of 4-hydroxyphenylglycine with CuCl₂ and O₂:

(comparison to Chem. Abstr. (1982): 597981)

In a 100 ml jacketed vessel, 5.0 g of 4-hydroxyphenylglycine (30 mmol) and 4.0 g of copper(II) chloride (30 mmol) were dissolved in 75 ml of water. At 90° C., oxygen was passed in (25 ml/min) and the reaction followed by means of HPLC. After 10 hours and at a conversion of 80%, only 6% by weight of product resulted, and in addition, 17.2% by weight of 4-hydroxybenzamide and a series of uncharacterized aromatic by-products were found.

1. A process for preparing substituted, aromatic aldehydes or ketones and optionally substituted, heteroaromatic aldehydes or ketones of the formula 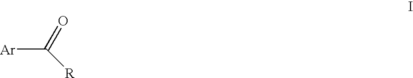
where Ar is a mono- or polysubstituted aromatic or an optionally mono- or polysubstituted heteroaromatic radical and the substituents are OH, methylenedioxy, C₁-C₆-alkyl, C₁-C₆-alkoxy, C₁-C₆-carboxylic acids or esters having 1-6 carbon atoms in the ester moiety, phenyl, halogen, SO₃H, NO₂, N₃, NR₁R₂ or SR₁ where R₁ and R₂ are each independently H, phenyl or C₁-C₆-alkyl, and R is H or an unsubstituted or polysubstituted alkyl group having 1-12 carbon atoms, unsubstituted or substituted benzyl or diphenylmethyl, and the substituents are heteroaromatics, halogens, NO₂, N₃, SO₃H, NR₁R₂ or SR₁ where R₁ and R₂ are each independently H, phenyl or C₁-C₆-alkyl, characterized in that a compound of the formula 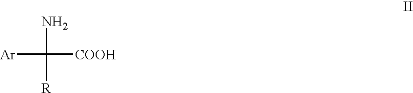
where Ar and R are each as defined above is converted in a suitable solvent in the presence of a carbonyl compound of the formula
R₃C(O)(CH═CH)ₙC(O)(X)_{y}R₃′ III
where n is 0 or an integer from 1 to 3, y is 0 or 1, X is NR₁, O or SO₂ and R₁ is H, C₁-C₆-alkyl or CH₂COOH, R₃ and R₃′ are each independently H, OH, C₁-C₆-alkyl, C₁-C₆-alkoxy or phenyl or together form a (—C═C—)ₘ bond where m is 1 or 2, or a fused ring system which is optionally substituted by OH, C₁-C₆-alkyl, C₁-C₆-alkoxy, C₁-C₆-carboxylic acids or esters having 1-6 carbon atoms in the ester moiety, phenyl, halogen, SO₃H, NO₂, N₃, NR₁R₂ or SR₁ where R₁ and R₂ are each independently H, phenyl or C₁-C₆-alkyl, and may optionally contain a heteroatom substituted by C₁-C₆-alkyl or CH₂COOH,
to the corresponding aldehyde or ketone of the formula I, optionally in the presence of oxygen, and at atmospheric or elevated pressure and at temperatures of from 5 to 200° C.

2. The process as claimed in claim 1, characterized in that Ar in the compound of the formulae I and II is a mono- or polysubstituted aromatic, an optionally mono- or polysubstituted heteroaromatic having one or more heteroatoms or an optionally mono- or polysubstituted fused ring system which optionally contains one or more heteroatoms, each of which is selected from the group of benzene, pyrrole, furan, thiophene, pyridine, pyran, thiopyran, pyrimidine, indene, imidazole, pyrazole, thiazole, oxazole, naphthalene, anthracene, quinoline, isoquinoline, benzo(g)isoquinoline, indole, coumaron, thionaphthene and acridine, and the substituents are selected from the group of OH, methylenedioxy, C₁-C₆-alkyl, C₁-C₆-alkoxy, phenyl, halogen, NR₁R₂ and SR₁ where R₁ and R₂ are each independently H, phenyl or C₁-C₆-alkyl and R is H or an unsubstituted or polysubstituted alkyl group having 1-12 carbon atoms, unsubstituted or substituted benzyl or diphenylmethyl, and the substituents are heteroaromatics, halogens, NO₂, N₃, SO₃H, NR₁R₂ or SR₁ where R₁ and R₂ are each independently H, phenyl or C₁-C₆-alkyl.

3. The process as claimed in claim 1, characterized in that the compound of the formula II used may be hydroxyphenylglycine, C₁-C₄-alkoxyphenylglycine, 3,4-dihydroxyphenylglycine, 2,4-dihydroxyphenylglycine, 3,4,5-trihydroxyphenylglycine, 3-hydroxy-4-methoxyphenylglycine, 4-hydroxy-3-methoxyphenylglycine, 3,4-methylenedioxyphenylglycine, α-methylphenylglycine, α-methyl-4-hydroxyphenylglycine, α-methyl-3,4-dihydroxyphenylglycine, α-methyl-3,4-methylenedioxyphenylglycine or α-methyl-4-methoxyphenylglycine.

4. The process as claimed in claim 1, characterized in that the solvent used is water or an aqueous mixture with organic and/or inorganic acids or with glycerol, dioxane or pyridine which have a pH of from 1 to 12.

5. The process as claimed in claim 1, characterized in that the carbonyl compound of the formula III used is an α-ketoacid, α-ketolactone, α-ketolactam, α-ketoaldehyde, 1,2-diketone, aldehydecarboxylic acid, dialdehyde, dicarboxylic acid or ester or quinone thereof.

6. The process as claimed in claim 1, characterized in that the carbonyl compound used is glyoxal, phenylglyoxal, methylglyoxal, diacetal, oxalic acid, maleic acid, glyoxylic acid, glyoxylic ester, phenylglyoxylic acid, pyruvic acid, pyruvic ester, phenylpyruvic acid, benzil, p-benzoquinone, o-benzoquinone, indigo, isatin, N-methylisatin, isatinsulfonic acid or isatinacetic acid.

7. The process as claimed in claim 1, characterized in that, depending on the type of the carbonyl compound, it is used here either in combination with oxygen in catalytic quantities of from 1 to 30 mol % or in an equimolar quantity, i.e. in a quantity of from 1.1 to 5 mol of carbonyl compound per mole of starting compound.

8. The process as claimed in claim 7, characterized in that isatin and substituted isatins are used in catalytic quantities of 1-20 mol %.

9. The process as claimed in claim 1, characterized in that when isatin or substituted isatins are used in the conversion of the compound of the formula II to the corresponding aldehyde or ketone of the formula I, oxygen is passed into the reaction mixture in the form of pure oxygen, air or an N₂/O₂ mixture.

10. The process as claimed in claim 1, characterized in that the reaction is carried out at a pressure of from 1 to 7 bar and reaction temperature of from 15 to 150° C.
